(19) United States
(12) Patent Application Publication
(10) Pub. No.: US 2008/0132971 A1
(43) Pub. Date: Jun. 5, 2008

(54) Electromagnetic apparatus for respiratory disease and method for using same

(76) Inventors: Arthur A. Pille, Oakland, NJ (US); Andre' Dimino, Woodcliff Lake, NJ (US)

Correspondence Address: LEN TAYLOR, PATENT ATTORNEY, 261 DAVENPORT STREET, SOMERVILLE, NJ, 08876, US

(21) Appl. No.: 11/903,294

(22) Filed: Sep. 20, 2007

Related U.S. Application Data
(60) Provisional application No. 60/846,126, filed on Sep. 20, 2006.

(51) Int. Cl. A61B 18/18

(52) U.S. Cl. 607/50

(57) ABSTRACT

A method for altering the electromagnetic environment of respiratory tissues, cells, and molecules comprising establishing baseline thermal fluctuations in voltage and electrical impedance at a respiratory target pathway structure depending on a state of the respiratory tissue, configuring at least one waveform to have sufficient signal to noise ratio to modulate at least one of ion and ligand interactions whereby the at least one of ion and ligand interactions are detectable in the respiratory target pathway structure above the established baseline thermal fluctuations in voltage and electrical impedance, generating an electromagnetic signal from the configured at least one waveform; and coupling the electromagnetic signal to the respiratory target pathway structure using a coupling device.

This application claims the benefit of U.S. Provisional Application 60/846,126 filed Sep. 20, 2006, herein incorporated by reference in its entirety.

### BACKGROUND OF THE INVENTION

### 1. Field of the Invention

This invention pertains to delivering electromagnetic signals to respiratory tissue such as lung tissue, of humans and animals that are injured or diseased whereby the interaction with the electromagnetic environment of living tissues, cells, and molecules is altered to achieve a therapeutic or wellness effect. The invention also relates to a method of modification of cellular and tissue growth, repair, maintenance and general behavior by the application of encoded electromagnetic information. More particularly, this invention provides for an application of highly specific electromagnetic frequency (“EMF”) signal patterns to lung tissue by surgically non-invasive reactive coupling of encoded electromagnetic information. Such application of electromagnetic waveforms to human and animal target pathway structures such as cells, organs, tissues and molecules, can serve to remedy injured or diseased respiratory tissue or to prophylactically treat such tissue.

The use of most low frequency EMF has been in conjunction with applications of bone repair and healing. As such, EMF waveforms and current orthopedic clinical use of EMF waveforms comprise relatively low frequency components inducing maximum electrical fields in a millivolts per centimeter (mV/cm) range at frequencies under five KHz. A linear physicochemical approach employing an electrochemical model of cell membranes to predict a range of EMF waveform patterns for which bioeffects might be expected is based upon an assumption that cell membranes, and specifically ion binding at structures in or on cell membranes or surfaces, are a likely EMF target. Therefore, it is necessary to determine a range of waveform parameters for which an induced electric field could couple electrochemically at a cellular surface, such as by employing voltage-dependent kinetics.

A pulsed radio frequency (“PRF”) signal derived from a 27.12 MHz continuous sine wave used for deep tissue healing is known in the prior art of diathermy. A pulsed successor of the diathermy signal was originally reported as an electromagnetic field capable of eliciting a non-thermal biological effect in the treatment of infections. Subsequently, PRF therapeutic applications have been reported for the reduction of post-traumatic and post-operative pain and edema in soft tissues, wound healing, burn treatment, and nerve regeneration. The application of PRF for resolution of traumatic and chronic edema has become increasingly used in recent years. Results to date using PRF in animal and clinical studies suggest that edema may be measurably reduced from such electromagnetic stimulus.

The within invention is based upon biophysical and animal studies that attribute effectiveness of cell-to-cell communication on tissue structures' sensitivity to induced voltages and associated currents. A mathematical power comparison analysis using at least one of a Signal to Noise Ratio (“SNR”) and a Power Signal to Noise Ratio (“Power SNR”) evaluates whether EMF signals applied to target pathway structures such as cells, tissues, organs, and molecules, are detectable above thermal noise present at an ion binding location. Prior art of EMF dosimetry did not take into account dielectric properties of tissue structures, rather the prior art utilized properties of isolated cells. By utilizing dielectric properties, reactive coupling of electromagnetic waveforms configured by optimizing SNR and Power SNR mathematical values evaluated at a target pathway structure can enhance wellness of the respiratory system as well as repair of various respiratory injuries and diseases in human and animal cells, organs, tissues and molecules for example sarcoidosis, granulomatous pneumonitis, pulmonary fibrosis, and “World Trade Center Cough.” Cell, organ, tissue, and molecule repair enhancement results from increased blood flow and anti-inflammatory effects, and modulation of angiogenesis and neovascularization as well as from other enhanced bioeffective processes such as growth factor and cytokine release.

Recent clinical use of non-invasive PRF at radio frequencies has used pulsed bursts of a 27.12 MHz sinusoidal wave, each pulse burst typically exhibiting a width of sixty five microseconds and having approximately 1,700 sinusoidal cycles per burst, and with various burst repetition rates.

Broad spectral density bursts of electromagnetic waveforms having a frequency in the range of one hertz (Hz) to one hundred megahertz (MHz), with 1 to 100,000 pulses per burst, and with a burst-repetition rate of 0.01 to 10,000 Hertz (Hz), are selectively applied to human and animal cells, organs, tissues and molecules. The voltage-amplitude envelope of each pulse burst is a function of a random, irregular, or other like variable, effective to provide a broad spectral density within the burst envelope. The variables are defined by mathematical functions that take into account signal to thermal noise ratio and Power SNR in specific target pathway structures. The waveforms are designed to modulate living cell growth, condition and repair. Particular applications of these signals include, but are not limited to, enhancing treatment of organs, muscles, joints, eyes, skin and hair, post surgical and traumatic wound repair, angiogenesis, improved blood perfusion, vasodilation, vasoconstriction, edema reduction, enhanced neovascularization, bone repair, tendon repair, ligament repair, organ regeneration and pain relief. The application of the within electromagnetic waveforms can serve to enhance healing of various respiratory tissue injuries and diseases, as well as provide prophylactic treatment for such tissue. The present invention is a non-invasive, non-pharmacological treatment modality that can have a salutary impact on persons suffering from respiratory diseases or conditions or that can be used on a prophylactic basis for those individuals who may be prone to respiratory diseases or conditions.

An aspect of the present invention is that a pulse burst envelope of higher spectral density can more efficiently couple to physiologically relevant dielectric pathways, such as cellular membrane receptors, ion binding to cellular enzymes, and general transmembrane potential changes. Another aspect of the present invention increases the number of frequency components transmitted to relevant cellular pathways, resulting in different electromagnetic characteristics of healing tissue and a larger range of biophysical phenomena applicable to known healing mechanisms becoming accessible, including enhanced enzyme activity, second messenger, such as nitric oxide (“NO”) release, growth factor release and cytokine release. By increasing burst duration and by applying a random, or other high spectral density envelope, to a pulse burst envelope of mono-polar or bi-polar rectangular or sinusoidal pulses that induce peak electric fields between 10⁻⁶ and 10 volts per centimeter (V/cm), and that satisfy detectability requirements according to SNR or Power SNR, a more efficient and greater effect could be achieved on biological healing processes applicable to both soft and hard tissues in humans and animals resulting in an acceleration of respiratory injury and disease repair.

The present invention relates to known mechanisms of respiratory injury and disease repair and healing that involve the naturally timed release of the appropriate anti-inflammatory cascade and growth factor or cytokine release in each stage of wound repair as applied to humans and animals. Specifically, respiratory injury and disease repair involves an inflammatory phase, angiogenesis, cell proliferation, collagen production, and remodeling stages. There are timed releases of second messengers, such as NO, specific cytokines and growth factors in each stage. Electromagnetic fields can enhance blood flow and enhance the binding of ions, which, in turn, can accelerate each healing phase. It is the specific intent of this invention to provide an improved means to enhance the action of endogenous factors and accelerate repair and to affect wellness. An advantageous result of using the present invention is that respiratory injury and disease repair, and healing can be accelerated due to enhanced blood flow or enhanced biochemical activity. In particular, an embodiment according to the present invention pertains to using an induction means such as a coil to deliver pulsing electromagnetic fields (“PEMF”) for the maintenance of the respiratory system and the treatment of respiratory diseases such sarcoidosis, granulomatous pneumonitis, pulmonary fibrosis, and “World Trade Center Cough”, and other related diseases. More particularly, this invention provides for the application, by surgically non-invasive reactive coupling, of highly specific electromagnetic signal patterns to one or more body parts. Such applications made on a non-invasive basis to the constituent tissues of the respiratory system and its surrounding tissues can serve to improve the physiological parameters of respiratory diseases.

Sarcoidosis, granulomatous pneumonitis, pulmonary fibrosis, and other related diseases result from inflammatory processes caused by inhalation of foreign material into lung tissue. The initiation of such diseases is the inflammation that occurs after particle inhalation. The within invention produces a physiological effect designed to reduce the inflammatory response, which in turn, may reduce the effects of inhaled foreign bodies on lung capacity and even prevent other systemic health problems. A number of physiological cascades that are accelerated or modified by the waveforms produced by the methods and apparatus of this invention serve to reduce the inflammatory processes. In particular, the PEMF signal can enhance the production of nitric oxide via modulation of Calcium (“Ca²⁺”) binding to calmodulin (“CaM”). This in turn can inhibit inflammatory leukotrienes that reduce the inflammatory process leading to excessive fibrous tissue for example scars, in lung tissue. Prophylactic use of the within invention by first responders may prevent or reduce the inflammatory processes leading to formation of fibrous tissue leading to lung disease.

Sarcoidosis involves inflammation that produces tiny agglomerations of cells in various organs of the body. These agglomerations are called glanulomas which are an aggregation and proliferation of macrophages to form nodules or granules. Such granulomas are of microscopic size and are not easily identifiable without significant magnification. Granulomas can grow and join together creating large and small groups of agglomerated cells. If there is a high prevalence of agglomerated granulomas in an organ, such as the lungs, the agglomerated granulomas can negatively impact the proper functioning of that organ. In the lungs, this negative impact can cause symptoms of sarcoidosis. Sarcoidosis can occur in almost any part of the body although it usually affects some organs such as the lungs and lymphnodes, more than others. It usually begins in one or two places, the lungs or lymphnodes especially the lymphnodes in the chest cavity. Sarcoidosis almost always occurs in more than one organ at a time. Exposure to pollutants or other particulates that are breathed into the lungs, such as dust and fibers present at the World Trade Center site after Sep. 11, 2001, can cause the scarring and resultant sarcoidosis.

Sarcoidosis involves both an active and a non-active phase. In the active phase, granulomas are formed and grow with symptoms developing. Scar tissue can form in the organs where such granulomas occur and inflammation is present. In the non-active phase, inflammation reduces, and the granulomas do not grow or may be reduced in size. If the non-active phase does occur, any scarring that occurred will remain and cause increased or continuing symptoms.

The course of the disease varies greatly. Sarcoidosis may be mild or severe. The inflammation that causes the granulomas may resolve without intervention and may stop growing or reduce in size. Symptoms may be reduced or alleviated within a few years after onset. In some cases, the inflammation remains but does not progress. There may be increased symptoms or flare-ups that require treatment on an intermittent basis. Although drug intervention can help, sarcoidosis may leave scar tissue in the lungs, skin, eyes or other organs and that scar tissue can permanently affect the functioning of the organs. Drug treatment usually does not affect scar tissue. The present invention has been shown in animal and clinical testing to reduce inflammation and accelerate angiogenesis and revascularization in organ tissue that may lead to improvement of vascularity of the tissue surrounding the scarring that may be the result of sarcoidosis in the lungs.

Sarcoidosis usually occurs slowly over many months and does not usually cause sudden illness. However, some symptoms may occur suddenly. These symptoms include disturbed heart rhythms, arthritis in the ankles, and eye symptoms. In some serious cases in which vital organs are affected, sarcoidosis can resulting death. However, sarcoidosis is not a form of cancer. Presently there is no way to prevent sarcoidosis. Sarcoidosis was once though to be an uncommon condition. It is now known to affect tens of thousands of people throughout the United States. Since many people who have sarcoidosis exhibit no symptoms, it is difficult to determine the actual prevalence of sarcoidosis in populations, although there seems to be a higher incidence in certain cultures.

An aspect of the present invention is to provide an improved means to accelerate the intended effects or improve efficacy as well as other effects of the second messengers, cytokines and growth factors relevant to each stage of respiratory injury and disease repair and healing.

Another aspect of the present invention is to cause and accelerate healing for treatment of respiratory diseases such as, sarcoidosis, granulomatous pneumonitis, pulmonary fibrosis, and “World Trade Center Cough” and other related diseases.

Another aspect of the present invention is to accelerate healing of respiratory injuries of any type.

Another aspect of the present invention is to maintain wellness of the respiratory system.

Another aspect of the present invention is that by applying a high spectral density voltage envelope as a modulating or pulse-burst defining parameter according to SNR and Power SNR requirements, power requirements for such increased duration pulse bursts can be significantly lower than that of shorter pulse bursts having pulses within the same frequency range; this results from more efficient matching of frequency components to a relevant cellular/molecular process. Accordingly, the advantage of enhanced transmitted dosimetry to relevant dielectric pathways and the advantage of decreased power requirements, are achieved. This advantageously allows for implementation of the within invention in an easily transportable unit for ease of application to the lung area and is particularly suitable for prophylactic use by first responders.

Another aspect of the present invention allows application of specific waveforms in a convenient and comfortable configuration to a desired pulmonary area. In an embodiment according to the present invention, a portable generator with multiple coil applicators that are incorporated into a body-conforming garment is worn by the user during a posteriori treatment or worn prophylactically. This allows for the proper positioning of the output coils to the chest area thereby allowing the produced signals to be broadcast over the lungs in an efficient manner.

Therefore, a need exists for an apparatus and a method that effectively enhances wellness of the respiratory system and accelerates healing of respiratory injuries, respiratory diseases, and areas around the respiratory system by modulating ion binding at cells, organs, tissues and molecules of humans and animals.

### SUMMARY OF THE INVENTION

The methods and apparatus according to present invention, comprises delivering electromagnetic signals to respiratory target pathway structures, such as respiratory molecules, respiratory cells, respiratory tissues, and respiratory organs for treatment of inflammatory processes leading to excessive fibrous tissue formation such as scar tissue, associated with the inhalation of foreign particles into lung tissue. An embodiment according to the present invention utilizes SNR and Power SNR approaches to configure bioeffective waveforms and incorporates miniaturized circuitry and lightweight flexible coils. This advantageously allows a device that utilizes the SNR and Power SNR approaches, miniaturized circuitry, and lightweight flexible coils to be completely portable and if desired to be constructed as disposable.

An embodiment according to the present invention comprises an electromagnetic signal having a pulse burst envelope of spectral density to efficiently couple to physiologically relevant dielectric pathways, such as cellular membrane receptors, ion binding to cellular enzymes, and general transmembrane potential changes. The use of a burst duration which is generally below 100 microseconds for each PRF burst, limits the frequency components that could couple to the relevant dielectric pathways in cells and tissue. An embodiment according to the present invention increases the number of frequency components transmitted to relevant cellular pathways whereby access to a larger range of biophysical phenomena applicable to known healing mechanisms, including enhanced second messenger release, enzyme activity and growth factor and cytokine release can be achieved. By increasing burst duration and applying a random, or other envelope, to the pulse burst envelope of mono-polar or bi-polar rectangular or sinusoidal pulses which induce peak electric fields between 10⁻⁶ and 10 V/cm, a more efficient and greater effect can be achieved on biological healing processes applicable to both soft and hard tissues in humans, animals and plants.

Another embodiment according to the present invention comprises known cellular responses to weak external stimuli such as heat, light, sound, ultrasound and electromagnetic fields. Cellular responses to such stimuli result in the production of protective proteins, for example, heat shock proteins, which enhance the ability of the cell, tissue, organ to withstand and respond to such external stimuli. Electromagnetic fields configured according to an embodiment of the present invention enhance the release of such compounds thus advantageously providing an improved means to enhance prophylactic protection and wellness of living organisms. In certain respiratory diseases there are physiological deficiencies and disease states that can have a lasting and deleterious effect on the proper functioning of the respiratory system. Those physiological deficiencies and disease states can be positively affected on a non-invasive basis by the therapeutic application of waveforms configured according to an embodiment of the present invention. In addition, electromagnetic waveforms configured according to an embodiment of the present invention can have a prophylactic effect on the respiratory system whereby a disease condition can be prevented, and if a disease condition already exists in its earliest stages, that condition can be prevented from developing into a more advanced state.

An example of a respiratory disease that can be positively affected by an embodiment according to the present invention, both on a chronic disease as well on a prophylactic basis, is inflammation in lung tissue resulting from inhalation of foreign particles that remain in lung tissue. Electromagnetic waveforms configured according to an embodiment of the present invention, have proven to have a positive effect on circulatory vessels and other tissues which can lead to reducing inflammation that can lead to lung disease.

Another advantage of electromagnetic waveforms configured according to an embodiment of the present invention is that by applying a high spectral density voltage envelope as the modulating or pulse-burst defining parameter, the power requirement for such increased duration pulse bursts can be significantly lower than that of shorter pulse bursts containing pulses within the same frequency range; this is due to more efficient matching of the frequency components to the relevant cellular/molecular process. Accordingly, the dual advantages, of enhanced transmitted dosimetry to the relevant dielectric pathways and of decreased power requirement are achieved.

The present invention relates to a therapeutically beneficial method of and apparatus for non-invasive pulsed electromagnetic treatment for enhanced condition, repair and growth of living tissue in animals, humans and plants. This beneficial method operates to selectively change the bioelectromagnetic environment associated with the cellular and tissue environment through the use of electromagnetic means such as PRF generators and applicator heads. An embodiment of the present invention more particularly includes the provision of a flux path, to a selectable body region, of a succession of EMF pulses having a minimum width characteristic of at least 0.01 microseconds in a pulse burst envelope having between 1 and 100,000 pulses per burst, in which a voltage amplitude envelope of said pulse burst is defined by a randomly varying parameter in which the instantaneous minimum amplitude thereof is not smaller than the maximum amplitude thereof by a factor of one ten-thousandth. Further, the repetition rate of such pulse bursts may vary from 0.01 to 10,000 Hz. Additionally a mathematically-definable parameter can be employed in lieu of said random amplitude envelope of the pulse bursts.

According to an embodiment of the present invention, by treating a selectable body region with a flux path comprising a succession of EMF pulses having a minimum width characteristic of at least about 0.01 microseconds in a pulse burst envelope having between about 1 and about 100,000 pulses per burst, in which a voltage amplitude envelope of said pulse burst is defined by a randomly varying parameter in which instantaneous minimum amplitude thereof is not smaller than the maximum amplitude thereof by a factor of one ten-thousandth. The pulse burst repetition rate can vary from about 0.01 to about 10,000 Hz. A mathematically definable parameter can also be employed to define an amplitude envelope of said pulse bursts.

By increasing a range of frequency components transmitted to relevant cellular pathways, access to a large range of biophysical phenomena applicable to known healing mechanisms, including enhanced second messenger release, enzyme activity and growth factor and cytokine release, is advantageously achieved.

According to an embodiment of the present invention, by applying a random, or other high spectral density envelope, to a pulse burst envelope of mono-polar or bi-polar rectangular or sinusoidal pulses which induce peak electric fields between 10⁻⁶ and 10 volts per centimeter (V/cm), a more efficient and greater effect can be achieved on biological healing processes applicable to both soft and hard tissues in humans, animals and plants. A pulse burst envelope of higher spectral density can advantageously and efficiently couple to physiologically relevant dielectric pathways, such as, cellular membrane receptors, ion binding to cellular enzymes, and general transmembrane potential changes thereby modulating angiogenesis and neovascularization.

Another advantage of an embodiment according to the present invention is that by applying a high spectral density voltage envelope as a modulating or pulse-burst defining parameter, power requirements for such modulated pulse bursts can be significantly lower than that of an unmodulated pulse. This is due to more efficient matching of the frequency components to the relevant cellular/molecular process. Accordingly, the dual advantages of enhanced transmitting dosimetry to relevant dielectric pathways and of decreasing power requirements are achieved.

An embodiment according to the present invention utilizes a Power Signal to Noise Ratio (“Power SNR”) approach to configure bioeffective waveforms and incorporates miniaturized circuitry and lightweight flexible coils. This advantageously allows a device that utilizes a Power SNR approach, miniaturized circuitry, and lightweight flexible coils, to be completely portable and if desired to be constructed as disposable and if further desired to be constructed as implantable. The lightweight flexible coils can be an integral portion of a positioning device such as surgical dressings, wound dressings, pads, seat cushions, mattress pads, wheelchairs, chairs, and any other garment and structure juxtaposed to living tissue and cells. By advantageously integrating a coil into a positioning device therapeutic treatment can be provided to living tissue and cells in an inconspicuous and convenient manner.

Specifically, broad spectral density bursts of electromagnetic waveforms, configured to achieve maximum signal power within a bandpass of a biological target, are selectively applied to target pathway structures such as living organs, tissues, cells and molecules. Waveforms are selected using a novel amplitude/power comparison with that of thermal noise in a target pathway structure. Signals comprise bursts of at least one of sinusoidal, rectangular, chaotic and random wave shapes have frequency content in a range of 0.01 Hz to 100 MHz at 1 to 100,000 bursts per second, with a burst duration from 0.01 to 100 milliseconds, and a burst repetition rate from 0.01 to 1000 bursts/second. Peak signal amplitude at a target pathway structure such as tissue, lies in a range of 1 μV/cm to 100 mV/cm. Each signal burst envelope may be a random function providing a means to accommodate different electromagnetic characteristics of healing tissue. Preferably the present invention comprises a 20 millisecond pulse burst, repeating at 1 to 10 burst/second and comprising 5 to 200 microsecond symmetrical or asymmetrical pulses repeating at 0.1 to 100 kilohertz within the burst. The burst envelope is a modified 1/f function and is applied at random repetition rates. Fixed repetition rates can also be used between about 0.1 Hz and about 1000 Hz. An induced electric field from about 0.001 mV/cm to about 100 mV/cm is generated. Another embodiment according to the present invention comprises a 4 millisecond of high frequency sinusoidal waves, such as 27.12 MHz, repeating at 1 to 100 bursts per second. An induced electric field from about 0.001 mV/cm to about 100 mV/cm is generated. Resulting waveforms can be delivered via inductive or capacitive coupling for 1 to 30 minute treatment sessions delivered according to predefined regimes by which PEMF treatment may be applied for 1 to 12 daily sessions, repeated daily. The treatment regimens for any waveform configured according to the instant invention may be fully automated. The number of daily treatments may be programmed to vary on a daily basis according to any predefined protocol.

In one aspect of the present invention, an electromagnetic method of treatment of living cells and tissues comprising a broad-band, high spectral density electromagnetic field is provided.

In another aspect of the present invention, an electromagnetic method of treatment of living cells and tissues comprising modulation of electromagnetically sensitive regulatory processes at a cell membrane and at junctional interfaces between cells is provided.

In another aspect of the present invention, an electromagnetic method of treatment of living cells and tissues comprising amplitude modulation of a pulse burst envelope of an electromagnetic signal that will induce coupling with a maximum number of relevant EMF-sensitive pathways in cells or tissues is provided.

In another aspect of the present invention, a power spectrum of a waveform is configured by mathematical simulation by using signal to noise ratio (“SNR”) analysis to configure a waveform optimized to modulate angiogensis and neovascularization then coupling the configured waveform using a generating device such as ultra lightweight wire coils that are powered by a waveform configuration device such as miniaturized electronic circuitry.

In another aspect of the present invention, multiple coils deliver a waveform configured by SNR/Power analysis of a target pathway structure, to increase area of treatment coverage.

In another aspect of the present invention, multiple coils that are simultaneously driven or that are sequentially driven such as multiplexed, deliver the same or different optimally configured waveforms as illustrated above.

In still another aspect of the present invention, flexible, lightweight coils that focus the EMF signal to the affected tissue delivering a waveform configured by SNR/Power analysis of a target pathway structure, are incorporated into dressings and ergonomic support garments.

In another aspect of the present invention, lightweight flexible coils or conductive thread is utilized to deliver the EMF signal to affected tissue by incorporating such coils or conductive threads as an integral part of various types of bandages, such as, compression, elastic, cold compress and hot compress and delivering a waveform configured by SNR/Power analysis of a target pathway structure.

In another aspect of the present invention, at least one coil is incorporated into a surgical wound dressing to apply an enhanced EMF signal non-invasively and non-surgically, the surgical wound dressing to be used in combination with standard wound treatment.

In another aspect of the present invention, the coils that deliver a waveform configured by SNR/Power analysis of a target pathway structure are constructed for easy attachment and detachment to dressings, garments and supports by using an attachment means such as Velcro®, an adhesive and any other such temporary attachment means.

In a further aspect of the present invention, at least one coil delivering a waveform configured by SNR/Power analysis of a target pathway structure, is integrated with a therapeutic surface, structure or device to enhance the effectiveness of such therapeutic surface, structure or device to augment the activity of cells and tissues of any type in any living target area.

In yet a further aspect of the present invention, an improved electromagnetic method of the beneficial treatment of living cells and tissue by the modulation of electromagnetically sensitive regulatory processes at the cell membrane and at junctional interfaces between cells is provided.

In a further aspect of the present invention, a means for the use of electromagnetic waveforms to cause a beneficial effect in the treatment of respiratory diseases is provided.

In a further aspect of the present invention, improved means for the prophylactic treatment of the respiratory system to improve function and to prevent or arrest diseases of the respiratory system is provided.

In another aspect of the present invention, an electromagnetic treatment method of the above type having a broad-band, high spectral density electromagnetic field is provided.

In a further aspect of the present invention, a method of the above type in which amplitude modulation of the pulse burst envelope of the electromagnetic signal will induce coupling with a maximum number of relevant EMF-sensitive pathways in cells or tissues is provided.

In another aspect of the present invention, an improved method of enhancing soft tissue and hard tissue repair is provided.

In another aspect of the present invention, an improved method of increasing blood flow to affected tissues by modulating angiogenesis is provided.

In another aspect of the present invention, an improved method of increasing blood flow to enhance the viability and growth or differentiation of implanted cells, tissues and organs is provided.

In another aspect of the present invention, an improved method of increasing blood flow in cardiovascular diseases by modulating angiogenesis is provided.

In another aspect of the present invention, beneficial physiological effects through improvement of micro-vascular blood perfusion and reduced transudation are provided.

In another aspect of the present invention, an improved method of treatment of maladies of the bone and other hard tissue is provided.

In still further aspect of the present invention, an improved means of the treatment of edema and swelling of soft tissue is provided.

In a further aspect of the present invention, an improved means to enhance second messenger release is provided.

In another aspect of the present invention, a means of repair of damaged soft tissue is provided.

In yet another aspect of the present invention, a means of increasing blood flow to damaged tissue by modulation of vasodilation and stimulating neovascularization is provided.

In yet a further aspect of the present invention, an apparatus that can operate at reduced power levels as compared to those of related methods known in electromedicine and respective biofield technologies, with attendant benefits of safety, economics, portability, and reduced electromagnetic interference is provided.

“About” for purposes of the invention means a variation of plus or minus 0.1%.

“Respiratory” for purposes of the invention means any organs and structures such as nose, nasal passages, nasopharynx, larynx, trachea, bronchi, lungs and airways in which gas exchange takes.

The above and yet other aspects and advantages of the present invention will become apparent from the hereinafter set forth Brief Description of the Drawings and Detailed Description of the Invention.

### BRIEF DESCRIPTION OF THE DRAWINGS

Methods and apparatus that are particular preferred embodiments of the invention will now be described, by way of example, with reference to the accompanying diagrammatic drawings:

FIG. 1 is a flow diagram of a method for altering an electromagnetic environment of respiratory tissue according to an embodiment of the present invention;

FIG. 2 is a view of an electromagnetic apparatus for respiratory tissue treatment according to an embodiment of the present invention;

FIG. 3 is a block diagram of miniaturized circuitry according to an embodiment of the present invention;

FIG. 4 depicts a waveform delivered to a respiratory target pathway structure according to an embodiment of the present invention;

FIG. 5 is a view of inductors placed in a vest according to an embodiment of the present invention;

FIG. 6 is a bar graph illustrating myosin phosphorylation for a PMF signal configured according to an embodiment of the present invention; and

FIG. 7 is a bar graph illustrating SNR signal effectiveness in a cell model of inflammation.

### DETAILED DESCRIPTION OF THE INVENTION

Induced time-varying currents from PEMF or PRF devices flow in a target pathway structure such as a molecule, cell, tissue, and organ, and it is these currents that are a stimulus to which cells and tissues can react in a physiologically meaningful manner. The electrical properties of a target pathway structure affect levels and distributions of induced current. Molecules, cells, tissue, and organs are all in an induced current pathway such as cells in a gap junction contact. Ion or ligand interactions at binding sites on macromolecules that may reside on a membrane surface are voltage dependent processes, that is electrochemical, that can respond to an induced electromagnetic field (“E”). Induced current arrives at these sites via a surrounding ionic medium. The presence of cells in a current pathway causes an induced current (“J”) to decay more rapidly with time (“J(t)”). This is due to an added electrical impedance of cells from membrane capacitance and ion binding time constants of binding and other voltage sensitive membrane processes such as membrane transport. Knowledge of ion binding time constants allows SNR to be evaluated for any EMF signal configuration. Preferably ion binding time constants in the range of about 1 to about 100 msec are used.

Equivalent electrical circuit models representing various membrane and charged interface configurations have been derived. For example, in Calcium (“Ca²⁺”) binding, the change in concentration of bound Ca²⁺ at a binding site due to induced E may be described in a frequency domain by an impedance expression such as:

$${Z}_{b} ( ω ) = {R}_{ion} + \frac{1}{iω {C}_{ion}}$$

which has the form of a series resistance-capacitance electrical equivalent circuit. Where ω is angular frequency defined as 2πf, where f is frequency, i=−1½, Zb(ω) is the binding impedance, and Rᵢₒₙ and Cᵢₒₙ are equivalent binding resistance and capacitance of an ion binding pathway. The value of the equivalent binding time constant, τᵢₒₙ=RᵢₒₙCᵢₒₙ, is related to a ion binding rate constant, k_{b}, via τᵢₒₙ=RᵢₒₙCᵢₒₙ=1/k_{b}. Thus, the characteristic time constant of this pathway is determined by ion binding kinetics.

Induced E from a PEMF or PRF signal can cause current to flow into an ion binding pathway and affect the number of Ca²⁺ ions bound per unit time. An electrical equivalent of this is a change in voltage across the equivalent binding capacitance Cᵢₒₙ, which is a direct measure of the change in electrical charge stored by Cᵢₒₙ. Electrical charge is directly proportional to a surface concentration of Ca²⁺ ions in the binding site that is storage of charge is equivalent to storage of ions or other charged species on cell surfaces and junctions. Electrical impedance measurements, as well as direct kinetic analyses of binding rate constants, provide values for time constants necessary for configuration of a PMF waveform to match a bandpass of target pathway structures. This allows for a required range of frequencies for any given induced E waveform for optimal coupling to target impedance, such as bandpass.

Ion binding to regulatory molecules is a frequent EMF target, for example Ca binding to calmodulin (“CaM”). Use of this pathway is based upon acceleration of tissue repair, for example bone repair, wound repair, hair repair, and repair of other molecules, cells, tissues, and organs that involves modulation of growth factors released in various stages of repair. Growth factors such as platelet derived growth factor (“PDGF”), fibroblast growth factor (“FGF”), and epidermal growth factor (“EGF”) are all involved at an appropriate stage of healing. Angiogenesis and neovascularization are also integral to tissue growth and repair and can be modulated by PMF. All of these factors are Ca/CaM-dependent.

Utilizing a Ca/CaM pathway a waveform can be configured for which induced power is sufficiently above background thermal noise power. Under correct physiological conditions, this waveform can have a physiologically significant bioeffect.

Application of a Power SNR model to Ca/CaM requires knowledge of electrical equivalents of Ca²⁺ binding kinetics at CaM. Within first order binding kinetics, changes in concentration of bound Ca²⁺ at CaM binding sites over time may be characterized in a frequency domain by an equivalent binding time constant, τᵢₒₙ=RᵢₒₙCᵢₒₙ, where Rᵢₒₙ and Cᵢₒₙ are equivalent binding resistance and capacitance of the ion binding pathway. τᵢₒₙ is related to a ion binding rate constant, k_{b}, via τᵢₒₙ=RᵢₒₙCᵢₒₙ=1/k_{b}. Published values for k_{b} can then be employed in a cell array model to evaluate SNR by comparing voltage induced by a PRF signal to thermal fluctuations in voltage at a CaM binding site. Employing numerical values for PMF response, such as Vmax=6.5×10−7 sec⁻¹, [Ca²⁺]=2.5 μM, KD=30 μM, [Ca²⁺CaM]=KD([Ca²⁺]+[CaM]), yields k_{b}=665 sec⁻¹ (τᵢₒₙ=1.5 msec). Such a value for τᵢₒₙ can be employed in an electrical equivalent circuit for ion binding while power SNR analysis can be performed for any waveform structure.

According to an embodiment of the present invention a mathematical model can be configured to assimilate that thermal noise is present in all voltage dependent processes and represents a minimum threshold requirement to establish adequate SNR. Power spectral density, Sn(ω), of thermal noise can be expressed as:

Sₙ(ω)=4kT Re[Z_{M}(x,ω)]

where Z_{M}(x,ω) is electrical impedance of a target pathway structure, x is a dimension of a target pathway structure and Re denotes a real part of impedance of a target pathway structure. Z_{M}(x,ω) can be expressed as:

$${Z}_{M} ( x , ω ) = [ \frac{{R}_{e} + {R}_{i} + {R}_{g}}{γ} ] tanh ( γ x )$$

This equation clearly shows that electrical impedance of the target pathway structure, and contributions from extracellular fluid resistance (“Re”), intracellular fluid resistance (“Ri”) and intermembrane resistance (“Rg”) which are electrically connected to target pathway structures all contribute to noise filtering.

A typical approach to evaluation of SNR uses a single value of a root mean square (RMS) noise voltage. This is calculated by taking a square root of an integration of Sₙ(ω)=4kT Re[Z_{M}(x,ω)] over all frequencies relevant to either a complete membrane response, or to bandwidth of a target pathway structure. SNR can be expressed by a ratio:

$$SNR = \frac{\vert {V}_{M} ( ω ) \vert}{RMS}$$

where |V_{M}(ω)| is maximum amplitude of voltage at each frequency as delivered by a chosen waveform to the target pathway structure.

An embodiment according to the present invention comprises a pulse burst envelope having a high spectral density, so that the effect of therapy upon the relevant dielectric pathways, such as, cellular membrane receptors, ion binding to cellular enzymes and general transmembrane potential changes, is enhanced. Accordingly by increasing a number of frequency components transmitted to relevant cellular pathways, a large range of biophysical phenomena, such as modulating growth factor and cytokine release and ion binding at regulatory molecules, applicable to known tissue growth mechanisms is accessible. According to an embodiment of the present invention applying a random, or other high spectral density envelope, to a pulse burst envelope of mono-polar or bi-polar rectangular or sinusoidal pulses inducing peak electric fields between about 10⁻⁸ and about 100 V/cm, produces a greater effect on biological healing processes applicable to both soft and hard tissues.

According to yet another embodiment of the present invention by applying a high spectral density voltage envelope as a modulating or pulse-burst defining parameter, power requirements for such amplitude modulated pulse bursts can be significantly lower than that of an unmodulated pulse burst containing pulses within a similar frequency range. This is due to a substantial reduction in duty cycle within repetitive burst trains brought about by imposition of an irregular amplitude and preferably a random amplitude onto what would otherwise be a substantially uniform pulse burst envelope. Accordingly, the dual advantages, of enhanced transmitted dosimetry to the relevant dielectric pathways and of decreased power requirement are achieved.

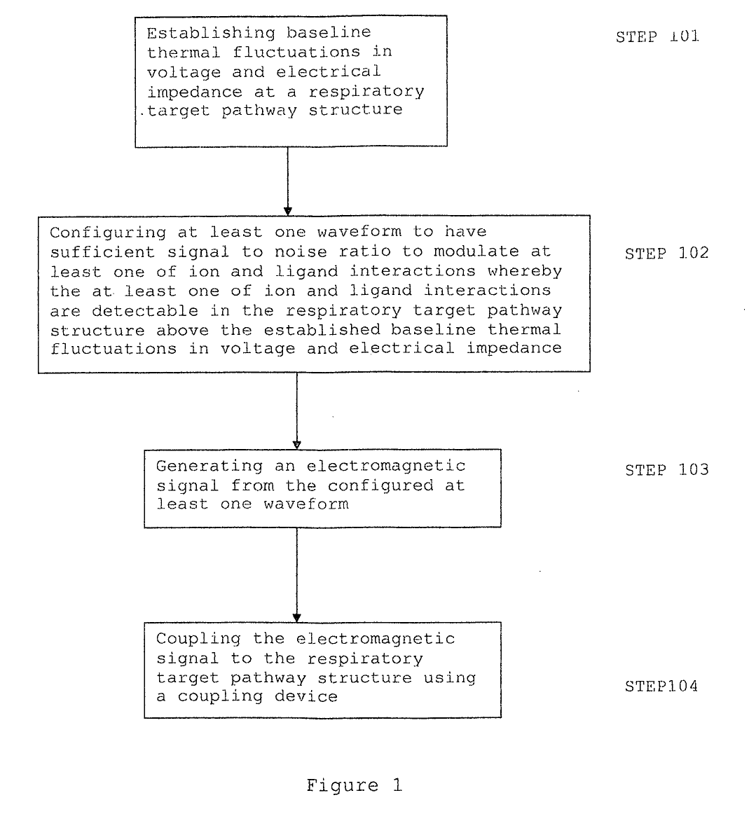

Referring to FIG. 1 wherein FIG. 1 is a flow diagram of a method for generating electromagnetic signals to be coupled to a respiratory target pathway structure according to an embodiment of the present invention, a target pathway structure such as ions and ligands, is identified. Establishing a baseline background activity such as baseline thermal fluctuations in voltage and electrical impedance, at the target pathway structure by determining a state of at least one of a cell and a tissue at the target pathway structure, wherein the state is at least one of resting, growing, replacing, and responding to injury. (STEP 101) The state of the at least one of a cell and a tissue is determined by its response to injury or insult. Configuring at least one waveform to have sufficient signal to noise ratio to modulate at least one of ion and ligand interactions whereby the at least one of ion and ligand interactions are detectable in the target pathway structure above the established baseline thermal fluctuations in voltage and electrical impedance. (STEP 102) Repetitively generating an electromagnetic signal from the configured at least one waveform. (STEP 103) The electromagnetic signal can be generated by using at least one waveform configured by applying a mathematical model such as an equation, formula, or function having at least one waveform parameter that satisfies an SNR or Power SNR mathematical model such that ion and ligand interactions are modulated and the at least one configured waveform is detectable at the target pathway structure above its established background activity. Coupling the electromagnetic signal to the target pathway structure using a coupling device. (STEP 104) The generated electromagnetic signals can be coupled for therapeutic and prophylactic purposes. The coupling enhances a stimulus that cells and tissues react to in a physiological meaningful manner for example, treatment of lung diseases resulting from inflammatory processes caused by inhalation of foreign material into lung tissue. Since lung tissue is very delicate, application of electromagnetic signals using an embodiment according to the present invention is extremely safe and efficient since the application of electromagnetic signals is non-invasive.

In an aspect of the present invention, a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model. A repetitive electromagnetic signal can be generated for example inductively or capacitively, from the configured at least one waveform. The electromagnetic signal is coupled to a target pathway structure such as ions and ligands by output of a coupling device such as an electrode or an inductor, placed in close proximity to the target pathway structure using a positioning device. The coupling enhances modulation of binding of ions and ligands to regulatory molecules, tissues, cells, and organs. According to an embodiment of the present invention EMF signals configured using SNR analysis to match the bandpass of a second messenger whereby the EMF signals can act as a first messenger to modulate biochemical cascades such as production of cytokines, Nitric Oxide, Nitric Oxide Synthase and growth factors that are related to tissue growth and repair. A detectable E field amplitude is produced within a frequency response of Ca²⁺ binding.

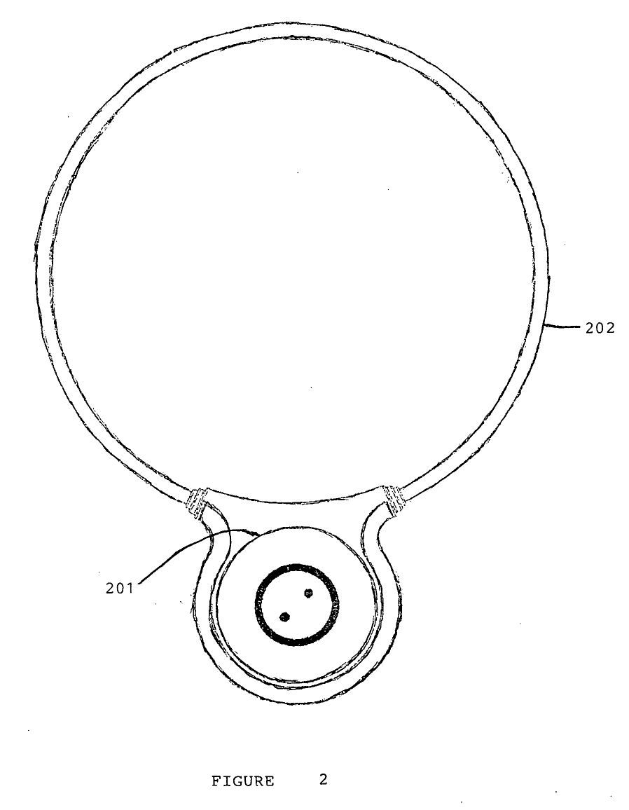

FIG. 2 illustrates an embodiment of an apparatus according to the present invention. The apparatus is self-contained, lightweight, and portable. A miniature control circuit 201 is connected to a generating device such as an electrical coil 202. The miniature control circuit 201 is constructed in a manner that applies a mathematical model that is used to configure waveforms. The configured waveforms have to satisfy a Power SNR model so that for a given and known target pathway structure, it is possible to choose waveform parameters that satisfy Power SNR so that a waveform is detectable in the target pathway structure above its background activity. An embodiment according to the present invention applies a mathematical model to induce a time-varying magnetic field and a time-varying electric field in a target pathway structure such as ions and ligands, comprising about 0.001 to about 100 msec bursts of about 1 to about 100 microsecond rectangular pulses repeating at about 0.1 to about 100 pulses per second. Peak amplitude of the induced electric field is between about 1 uV/cm and about 100 mV/cm, varied according to a modified 1/f function where f=frequency. A waveform configured using an embodiment according to the present invention may be applied to a target pathway structure such as ions and ligands, preferably for a total exposure time of under 1 minute to 240 minutes daily. However other exposure times can be used. Waveforms configured by the miniature control circuit 201 are directed to a generating device 202 such as electrical coils. Preferably, the generating device 202 is a comformable coil for example pliable, comprising one or more turns of electrically conducting wire in a generally circular or oval shape however other shapes can be used. The generating device 202 delivers a pulsing magnetic field configured according to a mathematical model that can be used to provide treatment to a target pathway structure such as lung tissue. The miniature control circuit applies a pulsing magnetic field for a prescribed time and can automatically repeat applying the pulsing magnetic field for as many applications as are needed in a given time period, for example 12 times a day. The miniature control circuit can be configured to be programmable applying pulsing magnetic fields for any time repetition sequence. An embodiment according to the present invention can be positioned to treat respiratory tissue by being incorporated with a positioning device such as a bandage or a vest thereby making the unit self-contained. Coupling a pulsing magnetic field to a target pathway structure such as ions and ligands, therapeutically and prophylactically reduces inflammation thereby reducing pain and promotes healing in treatment areas. When electrical coils are used as the generating device 202, the electrical coils can be powered with a time varying magnetic field that induces a time varying electric field in a target pathway structure according to Faraday's law. An electromagnetic signal generated by the generating device 202 can also be applied using electrochemical coupling, wherein electrodes are in direct contact with skin or another outer electrically conductive boundary of a target pathway structure. Yet in another embodiment according to the present invention, the electromagnetic signal generated by the generating device 202 can also be applied using electrostatic coupling wherein an air gap exists between a generating device 202 such as an electrode and a target pathway structure such as ions and ligands. An advantage of the present invention is that its ultra lightweight coils and miniaturized circuitry allow for use with common physical therapy treatment modalities, and at any location for which tissue growth, pain relief, and tissue and organ healing is desired. An advantageous result of application of the present invention is that tissue growth, repair, and maintenance can be accomplished and enhanced anywhere and at anytime. Yet another advantageous result of application of the present invention is that growth, repair, and maintenance of molecules, cells, tissues, and organs can be accomplished and enhanced anywhere and at anytime. Another embodiment according to the present invention delivers PEMF for application to respiratory tissue that is infected with diseases such as sarcoidosis, granulomatous pneumonitis, pulmonary fibrosis, and “World Trade Center Cough.”

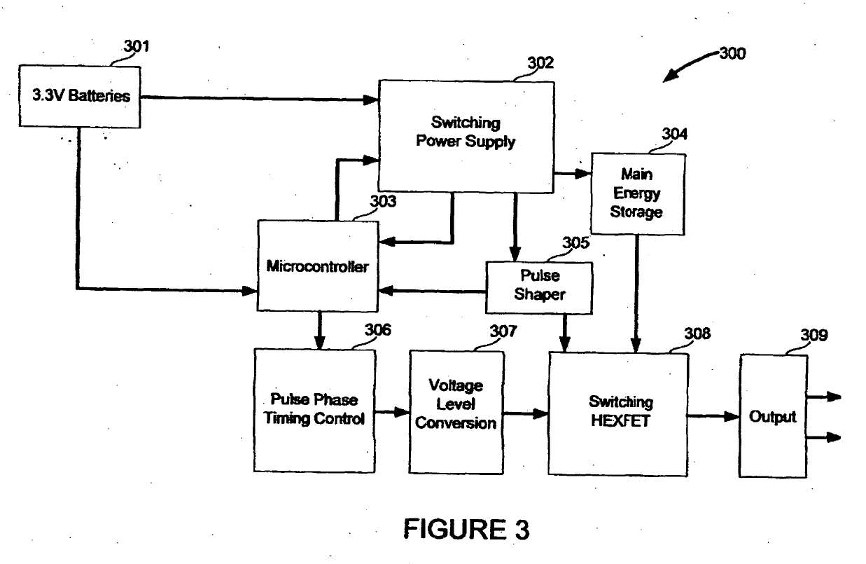

FIG. 3 depicts a block diagram of an embodiment according to the present invention of a miniature control circuit 300. The miniature control circuit 300 produces waveforms that drive a generating device such as wire coils described above in FIG. 2. The miniature control circuit can be activated by any activation means such as an on/off switch. The miniature control circuit 300 has a power source such as a lithium battery 301. Preferably the power source has an output voltage of 3.3 V but other voltages can be used. In another embodiment according to the present invention the power source can be an external power source such as an electric current outlet such as an AC/DC outlet, coupled to the present invention for example by a plug and wire. A switching power supply 302 controls voltage to a micro-controller 303. Preferably the micro-controller 303 uses an 8 bit 4 MHz micro-controller 303 but other bit MHz combination micro-controllers may be used. The switching power supply 302 also delivers current to storage capacitors 304. Preferably the storage capacitors 304 having a 220 uF output but other outputs can be used. The storage capacitors 304 allow high frequency pulses to be delivered to a coupling device such as inductors (Not Shown). The micro-controller 303 also controls a pulse shaper 305 and a pulse phase timing control 306. The pulse shaper 305 and pulse phase timing control 306 determine pulse shape, burst width, burst envelope shape, and burst repetition rate. In an aspect of the present invention the pulse shaper 305 and phase timing control 306 are configured such that the waveforms configured are detectable above background activity at a target pathway structure by satisfying at least one of a SNR and Power SNR mathematical model. An integral waveform generator, such as a sine wave or arbitrary number generator can also be incorporated to provide specific waveforms. A voltage level conversion sub-circuit 307 controls an induced field delivered to a target pathway structure. A switching Hexfet 308 allows pulses of randomized amplitude to be delivered to output 309 that routes a waveform to at least one coupling device such as an inductor. The micro-controller 303 can also control total exposure time of a single treatment of a target pathway structure such as a molecule, cell, tissue, and organ. The miniature control circuit 300 can be constructed to be programmable and apply a pulsing magnetic field for a prescribed time and to automatically repeat applying the pulsing magnetic field for as many applications as are needed in a given time period, for example 10 times a day. Preferably treatments times of about 1 minutes to about 30 minutes are used.

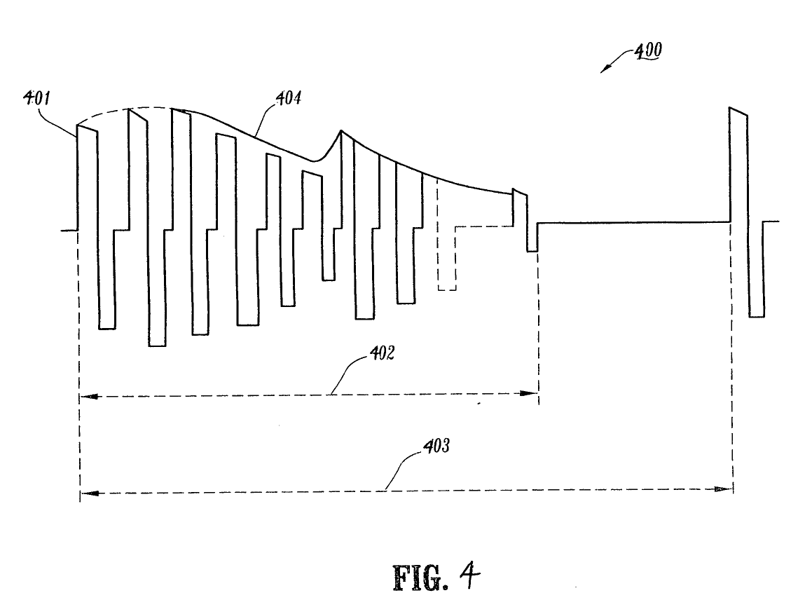

Referring to FIG. 4 an embodiment according to the present invention of a waveform 400 is illustrated. A pulse 401 is repeated within a burst 402 that has a finite duration or width 403. The duration 403 is such that a duty cycle which can be defined as a ratio of burst duration to signal period is between about 1 to about 10⁻⁵. Preferably pseudo rectangular 10 microsecond pulses for pulse 401 applied in a burst 402 for about 10 to about 50 msec having a modified 1/f amplitude envelope 404 and with a finite duration 403 corresponding to a burst period of between about 0.1 and about 10 seconds are utilized.

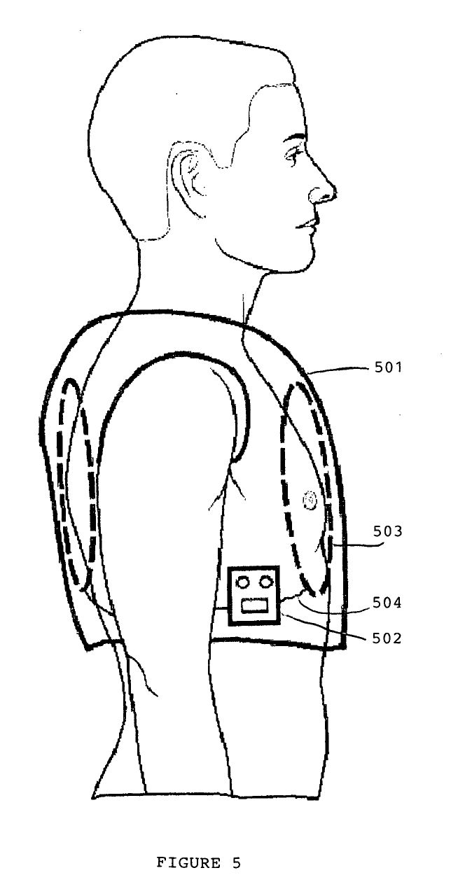

FIG. 5 illustrates an embodiment of an apparatus according to the present invention. A garment 501 such as a vest is constructed out of materials that are lightweight and portable such as nylon but other materials can be used. A miniature control circuit 502 is coupled to a generating device such as an electrical coil 503. Preferably the miniature control circuit 502 and the electrical coil 503 are constructed in a manner as described above in reference to FIG. 2. The miniature control circuit and the electrical coil can be connected with a connecting means such as a wire 504. The connection can also be direct or wireless. The electrical coil 503 is integrated into the garment 501 such that when a user wears the garment 501, the electrical coil is positioned near a lung or both lungs of the user. An advantage of the present invention is that its ultra lightweight coils and miniaturized circuitry allow for the garment 501 to be completely self-contained, portable, and lightweight. An additionally advantageous result of the present invention is that the garment 501 can be constructed to be inconspicuous when worn and can be worn as an outer garment such as a shirt or under other garments, so that only the user will know that the garment 501 is being worn and treatment is being applied. Use with common physical therapy treatment modalities, and at any respiratory location for which tissue growth, pain relief, and tissue and organ healing is easily obtained. An advantageous result of application of the present invention is that tissue growth, repair, and maintenance can be accomplished and enhanced anywhere and at anytime. Yet another advantageous result of application of the present invention is that growth, repair, and maintenance of molecules, cells, tissues, and organs can be accomplished and enhanced anywhere and at anytime. Another embodiment according to the present invention delivers PEMF for application to respiratory tissue that is infected with diseases such as sarcoidosis, granulomatous pneumonitis, pulmonary fibrosis, and “World Trade Center Cough.”

It is further intended that any other embodiments of the present invention that result from any changes in application or method of use or operation, method of manufacture, shape, size or material which are not specified within the detailed written description or illustrations and drawings contained herein, yet are considered apparent or obvious to one skilled in the art, are within the scope of the present invention.

The process of the invention will now be described with reference to the following illustrative examples.

### EXAMPLE 1

The Power SNR approach for PMF signal configuration has been tested experimentally on calcium dependent myosin phosphorylation in a standard enzyme assay. The cell-free reaction mixture was chosen for phosphorylation rate to be linear in time for several minutes, and for sub-saturation Ca²⁺ concentration. This opens the biological window for Ca²⁺/CaM to be EMF-sensitive. This system is not responsive to PMF at levels utilized in this study if Ca²⁺ is at saturation levels with respect to CaM, and reaction is not slowed to a minute time range. Experiments were performed using myosin light chain (“MLC”) and myosin light chain kinase (“MLCK”) isolated from turkey gizzard. A reaction mixture consisted of a basic solution containing 40 mM Hepes buffer, pH 7.0; 0.5 mM magnesium acetate; 1 mg/ml bovine serum albumin, 0.1% (w/v) Tween80; and 1 mM EGTA12. Free Ca²⁺ was varied in the 1-7 μM range. Once Ca²⁺ buffering was established, freshly prepared 70 nM CaM, 160 nM MLC and 2 nM MLCK were added to the basic solution to form a final reaction mixture. The low MLC/MLCK ratio allowed linear time behavior in the minute time range. This provided reproducible enzyme activities and minimized pipetting time errors.

The reaction mixture was freshly prepared daily for each series of experiments and was aliquoted in 100 μL portions into 1.5 ml Eppendorf tubes. All Eppendorf tubes containing reaction mixture were kept at 0° C. then transferred to a specially designed water bath maintained at 37±0.1° C. by constant perfusion of water prewarmed by passage through a Fisher Scientific model 900 heat exchanger. Temperature was monitored with a thermistor probe such as a Cole-Parmer model 8110-20, immersed in one Eppendorf tube during all experiments. Reaction was initiated with 2.5 μM 32P ATP, and was stopped with Laemmli Sample Buffer solution containing 30 μM EDTA. A minimum of five blank samples were counted in each experiment. Blanks comprised a total assay mixture minus one of the active components Ca²⁺, CaM, MLC or MLCK. Experiments for which blank counts were higher than 300 cpm were rejected. Phosphorylation was allowed to proceed for 5 min and was evaluated by counting 32P incorporated in MLC using a TM Analytic model 5303 Mark V liquid scintillation counter.

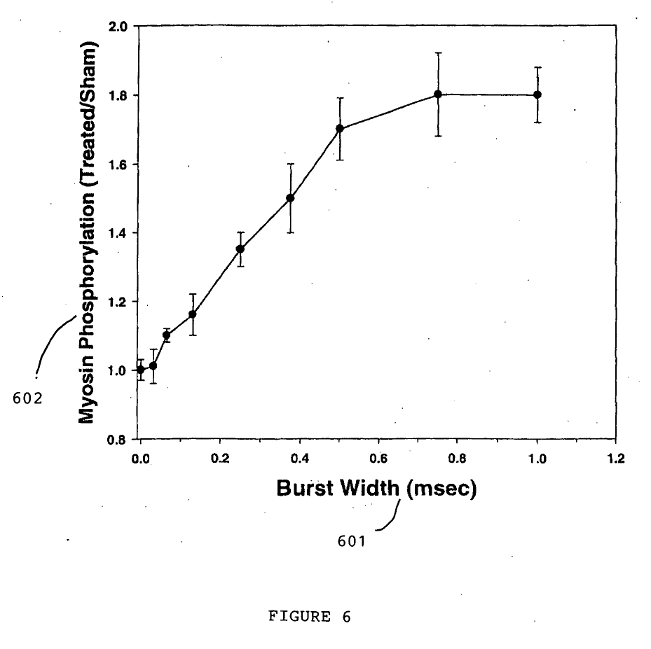

The signal comprised repetitive bursts of a high frequency waveform. Amplitude was maintained constant at 0.2 G and repetition rate was 1 burst/sec for all exposures. Burst duration varied from 65 μsec to 1000 μsec based upon projections of Power SNR analysis which showed that optimal Power SNR would be achieved as burst duration approached 500 μsec. The results are shown in FIG. 6 wherein burst width 601 in msec is plotted on the x-axis and Myosin Phosphorylation 602 as treated/sham is plotted on the y-axis. It can be seen that the PMF effect on Ca²⁺ binding to CaM approaches its maximum at approximately 500 μsec, just as illustrated by the Power SNR model.

These results confirm that a PMF signal, configured according to an embodiment of the present invention, would maximally increase myosin phosphorylation for burst durations sufficient to achieve optimal Power SNR for a given magnetic field amplitude.

### EXAMPLE 2

According to an embodiment of the present invention use of a Power SNR model was further verified in an in vivo wound repair model. A rat wound model has been well characterized both biomechanically and biochemically, and was used in this study. Healthy, young adult male Sprague Dawley rats weighing more than 300 grams were utilized.

The animals were anesthetized with an intraperitoneal dose of Ketamine 75 mg/kg and Medetomidine 0.5 mg/kg. After adequate anesthesia had been achieved, the dorsum was shaved, prepped with a dilute betadine/alcohol solution, and draped using sterile technique. Using a #10 scalpel, an 8-cm linear incision was performed through the skin down to the fascia on the dorsum of each rat. The wound edges were bluntly dissected to break any remaining dermal fibers, leaving an open wound approximately 4 cm in diameter. Hemostasis was obtained with applied pressure to avoid any damage to the skin edges. The skin edges were then closed with a 4-0 Ethilon running suture. Post-operatively, the animals received Buprenorphine 0.1-0.5 mg/kg, intraperitoneal. They were placed in individual cages and received food and water ad libitum.

PMF exposure comprised two pulsed radio frequency waveforms. The first was a standard clinical PRF signal comprising a 65 μsec burst of 27.12 MHz sinusoidal waves at 1 Gauss amplitude and repeating at 600 bursts/sec. The second was a PRF signal reconfigured according to an embodiment of the present invention. For this signal burst duration was increased to 2000 μsec and the amplitude and repetition rate were reduced to 0.2 G and 5 bursts/sec respectively. PRF was applied for 30 minutes twice daily.

Tensile strength was performed immediately after wound excision. Two 1 cm width strips of skin were transected perpendicular to the scar from each sample and used to measure the tensile strength in kg/mm2. The strips were excised from the same area in each rat to assure consistency of measurement. The strips were then mounted on a tensiometer. The strips were loaded at 10 mm/min and the maximum force generated before the wound pulled apart was recorded. The final tensile strength for comparison was determined by taking the average of the maximum load in kilograms per mm2 of the two strips from the same wound.

The results showed average tensile strength for the 65 μsec 1 Gauss PRF signal was 19.3±4.3 kg/mm2 for the exposed group versus 13.0±3.5 kg/mm2 for the control group (p<0.01), which is a 48% increase. In contrast, the average tensile strength for the 2000 μsec 0.2 Gauss PRF signal, configured according to an embodiment of the present invention using a Power SNR model was 21.2±5.6 kg/mm2 for the treated group versus 13.7±4.1 kg/mm2 (p<0.01) for the control group, which is a 54% increase. The results for the two signals were not significantly different from each other.

These results demonstrate that an embodiment of the present invention allowed a new PRF signal to be configured that could be produced with significantly lower power. The PRF signal configured according to an embodiment of the present invention, accelerated wound repair in the rat model in a low power manner versus that for a clinical PRF signal which accelerated wound repair but required more than two orders of magnitude more power to produce.

### EXAMPLE 3

This example illustrates the effects of PMF stimulation of a T-cell receptor with cell arrest and thus behave as normal T-lymphocytes stimulated by antigens at the T-cell receptor such as anti-CD3.

In bone healing, results have shown that both 60 Hz and PEMF fields decrease DNA synthesis of Jurkat cells, as is expected since PMF interacts with the T-cell receptor in the absence of a costimulatory signal. This result is consistent with an anti-inflammatory response, as has been observed in clinical applications of PMF stimuli. The PEMF signal is more effective. A dismetry analysis performed according to an embodiment of the present invention demonstrates why both signals are effective and why PEMF signals have a greater effect than 60 Hz signals on Jurkat cells in the most EMF-sensitive growth stage.

Comparison of dosimetry from the two signals employed involves evaluation of the ratio of the Power spectrum of the thermal noise voltage that is Power SNR, to that of the induced voltage at the EMF-sensitive target pathway structure. The target pathway structure used is ion binding at receptor sites on Jurkat cells suspended in 2 mm of culture medium. The average peak electric field at the binding site from a PEMF signal comprising 5 msec burst of 200 μsec pulses repeating at 15/sec was 1 mV/cm, while for a 60 Hz signal the average peak electric field was 100 μV/cm.

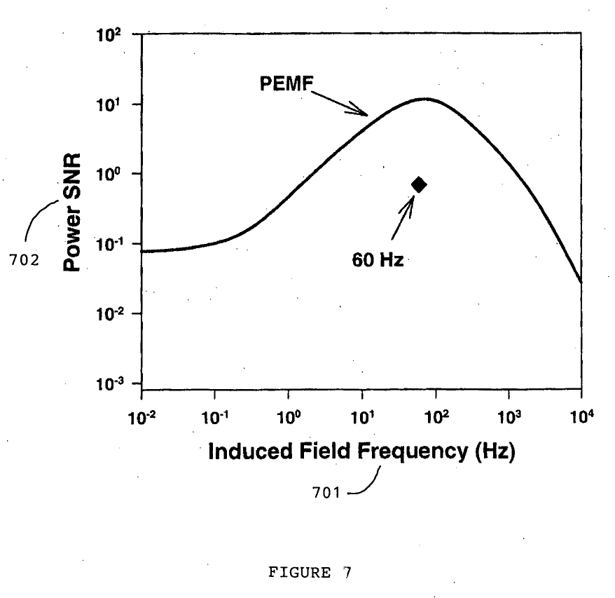

FIG. 7, is a graph of results wherein Induced Field Frequency 701 in Hz is shown on the x-axis and Power SNR 702 is shown on the y-axis. FIG. 7 illustrates that both signals have sufficient Power spectrum that is Power SNR≧1, to be detected within a frequency range of binding kinetics. However, maximum Power SNR for the PEMF signal is significantly higher than that of the 60 Hz signal. This is due to a PEMF signal having many frequency components falling within a bandpass of the target pathway structure. The single frequency component of a 60 Hz signal lies at the mid-point of the bandpass of a target pathway structure. The Power SNR calculation that was used in this example is dependent upon τᵢₒₙ which is obtained from the rate constant for ion binding. Had this calculation been performed a priori it would have concluded that both signals satisfied basic detectability requirements and could modulate an EMF-sensitive ion binding pathway at the start of a regulatory cascade for DNA synthesis in these cells. The previous examples illustrate that utilizing the rate constant for Ca/CaM binding could lead to successful projections for bioeffective EMF signals in a variety of systems.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

What is claimed is:

1) An electromagnetic apparatus comprising:
an electromagnetic signal generating means for emitting signals comprising bursts of at least one of sinusoidal, rectangular, chaotic, and random waveforms, having a frequency content in a range of about 0.01 Hz to about 100 MHz at about 1 to about 100,000 waveforms per second, having a burst duration of 1 usec to 100 msec, and having a burst repetition rate from about 0.01 to about 1000 bursts/second, wherein the waveforms are adapted to have sufficient signal to noise ratio in respect of a given respiratory target pathway structure to modulate at least one of ion and ligand interactions in that respiratory target pathway structure,
an electromagnetic signal coupling means wherein the coupling means comprises at least one of an inductive coupling means and a capacitive coupling means, connected to the electromagnetic signal generating means for delivering the electromagnetic signal to the respiratory target pathway structure, and
a chest garment wherein the electromagnetic signal generating means and electromagnetic signal coupling means are incorporated into the chest garment.

2) The apparatus of claim 1, wherein the signals comprise about 0.001 to about 100 msec bursts repeating at about 0.1 to about 10 pulses per second of about 1 to about 100 microsecond rectangular pulses.

3) The apparatus of claim 1, configured for providing an emitted signal having an peak signal amplitude at a respiratory target pathway structure in a range of about 1 μV/cm to about 100 mV/cm.

4) The apparatus of claim 1, wherein each signal burst envelope is a random function for providing a means to accommodate different electromagnetic characteristics of healing tissue.

5) The apparatus of claim 1, wherein the apparatus is configured for emitting a 20 millisecond pulse burst comprising about 0.1 microsecond to about 20 microsecond at least one of symmetrical and asymmetrical pulses repeating at about 1 to about 100 KHz within the burst.

6) The apparatus of claim 1, wherein the apparatus is configured for emitting an about 1 millisecond to an about 5 millisecond burst of 27.12 MHz sinusoidal waves repeating at about 1 to about 100 bursts/sec.

7) An electromagnetic apparatus comprising:
A waveform configuration means for configuring at least one waveform to have sufficient signal to noise ratio or power signal to noise ratio to modulate at least one of ion and ligand interactions whereby the at least one of ion and ligand interactions are detectable in a respiratory target pathway structure above baseline thermal fluctuations in voltage and electrical impedance at the respiratory target pathway structure;
A coupling device connected to the waveform configuration means by at least one connecting means for generating an electromagnetic signal from the configured at least one waveform and for coupling the electromagnetic signal to the respiratory target pathway structure whereby the at least one of ion and ligand interactions are modulated; and
A chest garment incorporating the waveform configuration means, the at least one connecting means, and the coupling device.

8) The apparatus of claim 7, wherein the at least one of ion and ligand interactions includes at least one of calcium ion binding and binding of calcium ions to calmodulin.

9) The apparatus of claim 7, wherein the configuration means includes a configuration means for configuring at least one waveform having at least one of a frequency component parameter that configures said at least one waveform to be between about 0.01 Hz and about 100 MHz, a burst amplitude envelope parameter that follows an arbitrary amplitude function, a burst amplitude envelope parameter that follows a defined amplitude function, a burst width parameter that varies at each repetition according to an arbitrary width function, a burst width parameter that varies at each repetition according to a defined width function, a peak induced electric field parameter varying between about 1 μV/cm and about 100 mV/cm in said target pathway structure, and a peak induced magnetic electric field parameter varying between about 1 μT and about 0.1 T in said target pathway structure.

10) The apparatus of claim 9, wherein said defined amplitude function includes at least one of a 1/frequency function, a logarithmic function, a chaotic function and an exponential function.

11) The apparatus of claim 7, wherein said coupling device includes at least one of an inductive generating coupling device, a capacitive generating coupling device, an inductor, and an electrode.

12) The apparatus of claim 7, wherein said chest garment includes at least one of a vest, jacket, shirt, and coat.

13) The apparatus of claim 7, wherein at least one of said waveform configuration means, connecting means, and coupling device is at least one of portable, disposable, implantable, and wireless.

14) A method for altering the electromagnetic environment of respiratory tissues, cells, and molecules comprising:
Establishing baseline thermal fluctuations in voltage and electrical impedance at a respiratory target pathway structure depending on a state of the respiratory tissue,
Configuring at least one waveform to have sufficient signal to noise ratio to modulate at least one of ion and ligand interactions whereby the at least one of ion and ligand interactions are detectable in the respiratory target pathway structure above the established baseline thermal fluctuations in voltage and electrical impedance;
Generating an electromagnetic signal from the configured at least one waveform; and
Coupling the electromagnetic signal to the respiratory target pathway structure using a coupling device.

15) The method of claim 14, wherein the step of configuring at least one waveform to have sufficient signal to noise ratio to modulate at least one of ion and ligand interactions includes configuring at least one waveform to have sufficient signal to noise ratio to modulate calcium ion binding.

16) The method of claim 14, wherein the step of configuring at least one waveform to have sufficient signal to noise ratio to modulate at least one of ion and ligand interactions includes configuring at least one waveform to have sufficient signal to noise ratio to modulate binding of calcium ions to calmodulin.

17) The method of claim 14, wherein the step of configuring at least one waveform to have sufficient signal to noise ratio to modulate at least one of ion and ligand interactions includes configuring at least one waveform to have sufficient signal to noise ratio to match a bandpass of a second messenger at a respiratory target pathway structure whereby the second messenger modulates biochemical cascades related to tissue growth and repair.

18) The method of claim 14, wherein the step of establishing baseline thermal fluctuations in voltage and electrical impedance at a respiratory target pathway structure includes establishing baseline thermal fluctuations in voltage and electrical impedance at least one of a respiratory molecule, a respiratory cell, a respiratory tissue, and a respiratory organ.

19) The method of claim 14, wherein the step of coupling the electromagnetic signal to the respiratory target pathway structure using a coupling device includes coupling the electromagnetic signal to the respiratory target pathway structure using at least one of an inductive generating coupling device, a capacitive generating coupling device, an inductor, and an electrode.

20) The method of claim 14, wherein the step of coupling the electromagnetic signal to the respiratory target pathway structure includes coupling the electromagnetic signal to the respiratory target pathway structure to enhance the production of second messengers at the respiratory target pathway structure.

21) The method of claim 20, wherein the step of coupling the electromagnetic signal to the respiratory target pathway structure to enhance the production of second messengers at the respiratory target pathway structure includes coupling the electromagnetic signal to the respiratory target pathway structure to enhance the production of Nitric Oxide at the respiratory target pathway structure.

22) The method of claim 14, wherein the step of coupling the electromagnetic signal to the respiratory target pathway structure includes coupling the electromagnetic signal to the respiratory target pathway structure to enhance the production of growth factors at the respiratory target pathway structure.

23) The method of claim 14, wherein the step of coupling the electromagnetic signal to the respiratory target pathway structure includes coupling the electromagnetic signal to the respiratory target pathway structure to enhance the production of cytokines at the respiratory target pathway structure.

24) The method of claim 14, wherein the step of coupling the electromagnetic signal to the respiratory target pathway structure includes coupling the electromagnetic signal to the respiratory target pathway structure to enhance modulation of binding of at least one of ions and ligands to at least one of regulatory molecules, tissues, cells, and organs.

25) The method of claim 14, wherein the step of coupling the electromagnetic signal to the respiratory target pathway structure includes coupling the electromagnetic signal to the respiratory target pathway structure to provide treatment for at least one of sarcoidosis, granulomatous pneumonitis, pulmonary fibrosis, and “World Trade Center Cough.”
